(19) United States
(12) Patent Application Publication
(10) Pub. No.: US 2003/0225294 A1
(43) Pub. Date: Dec. 4, 2003

(54) Process for the preparation of N,N' -disubstituted 1,4-diaminoanthraquinones

(76) Inventors: Ulrich Feldhues, Bergisch Gladbach (DE); Josef-Walter Stawitz, Odenthal (DE); Julius Naseband, Odenthal (DE)

Correspondence Address: BAYER CHEMICALS CORPORATION, 100 BAYER ROAD, PITTSBURGH, PA, 15205, US

(21) Appl. No.: 10/439,995

(22) Filed: May 16, 2003

(30) Foreign Application Priority Data
May 21, 2002 (DE) 10222819.1

(51) Int. Cl. C08L 1/00; C09B 1/16; C07C 225/34

(52) U.S. Cl. 552/236; 524/242

(57) ABSTRACT

Process for the preparation of N,N′-disubstituted 1,4-diaminoanthraquinones

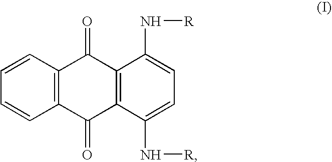

in which

R is an aliphatic or aromatic radical,

characterized in that 1,4-dihydroxyanthraquinones are reacted with aliphatic or aromatic amines in the presence of a readily water-soluble, dipolar-aprotic solvent (e.g. NMP) and of a second solvent, different from the first.

### BACKGROUND OF THE INVENTION

The invention relates to a process for the preparation of N,N′-disubstituted 1,4-diaminoanthraquinones and also to the use of compounds thus prepared for the mass coloration of plastics.

N,N′-disubstituted 1,4-diaminoanthraquinones are known, for example, as dyes for plastics and synthetic fibres and for precursors for the preparation of wool dyes. Hitherto, 1,4-diaminoanthraquinones have been prepared by reacting 1,4-dihydroxyanthraquinone (quinizarine), optionally in a mixture with 2,3-dihydro-1,4-dihydroxyanthraquinone (leucoquinizarine), with amines, the reaction having been carried out optionally in the presence of condensation auxiliaries. Examples of known condensation auxiliaries are hydrochloric acid (DE-A-2 342 469), acetic acid (U.S. Pat. No. 4,083,683) or hydroxy carboxylic acids (DE-A-195 17 071). Even when using these auxiliaries, however, neither the reaction times nor the yields are optimal. According to the prior art, byproducts are formed, some of which are insoluble in the reaction medium, and which then may turn up again as insoluble impurities in the main product. A further disadvantage are considerable viscosity problems in the course of the reaction, which lead to poor space/time yields.

DE-A 199 36 282 describes the preparation of aminohydroxyanthraquinones from chlorohydroxyanthraquinones, inter alia, in the presence of readily water-soluble, dipolar-aprotic solvents. Under these conditions the hydroxyl group is not replaced by an amino group.

It is an object of the present invention to provide a process which no longer possesses the disadvantages of the prior art.

### SUMMARY OF THE INVENTION

A process has now been found for the preparation of N,N′-disubstituted 1,4-diaminoanthraquinones which is characterized in that 1,4-dihydroxyanthraquinone (quinizarine) is reacted with aliphatic or aromatic amines in the presence of a dipolar-aprotic solvent having a water solubility of at least 30% by weight at 20° C., based on the aqueous solution, and at least one second solvent, different from the first.

### DETAILED DESCRIPTION OF THE INVENTION

The dipolar-aprotic solvent is preferably miscible with water. Preference is given to optionally substituted formamide or sulphoxide such as dimethylformamide, dimethyl sulphoxide, an optionally substituted lactam or lactone, with particular preference butyrolactone, N-methyl-2-pyrrolidone or caprolactam, with very particular preference N-methyl-2-pyrrolidone.

The second solvent preferably possesses a solubility in water of less than 20% by weight at 20° C., based on the aqueous solution, with particular preference for those possessing a solubility in water of from 5% by weight to 15% by weight at 20° C. The second solvent preferably forms an azeotrope with water. The second solvent is preferably miscible with the dipolar-aprotic solvent. Examples of suitable second solvents are aliphatic alcohols, such as 1-butanol, 2-methyl-1-propanol, 2-butanol, and also optionally substituted aromatics such as dichlorobenzene, toluene, xylene, etc. Amine used in excess can also be employed as second solvent. Particular preference is given to protic solvents and very particular preference to aliphatic alcohols, especially 1-butanol.

The dipolar-aprotic solvent and the second solvent are preferably used in a ratio of from 2:98 to 98:2, in particular from 5:95 to 50:50, with very particular preference from 10:90 to 20:80.

The 1,4-dihydroxyanthraquinone (quinizarine) used in the process of the invention is employed preferably in a mixture with its leuco form, 2,3-dihydro-1,4-dihydroxyanthraquinone (leucoquinizarine), preferably in a ratio of from 95:5 to 25:75, in particular from 90:10 to 50:50, with very particular preference from 90:10 to 70:30.

The mixture of leucoquinizarine and quinizarine may be formed, for example, in situ from the quinizarine by adding reducing agents such as zinc dust or sodium dithionite. The anthraquinone compounds, quinizarine and its leuco form, may alternatively be prepared separately.

In one preferred variant the leuco form is not generated or added until during the course of the reaction. It is particularly preferred to add the leuco form in solution in the dipolar-aprotic solvent. It is particularly advantageous to add the leuco form to the hot reaction mixture which has already been heated, preferably to more than 500° C.

The preferred aliphatic or aromatic amines employed in the process of the invention are primary. The aliphatic amines may, for example, be saturated, unsaturated, branched or straight-chain. Examples of particularly preferred aliphatic amines are those of the following formulae:

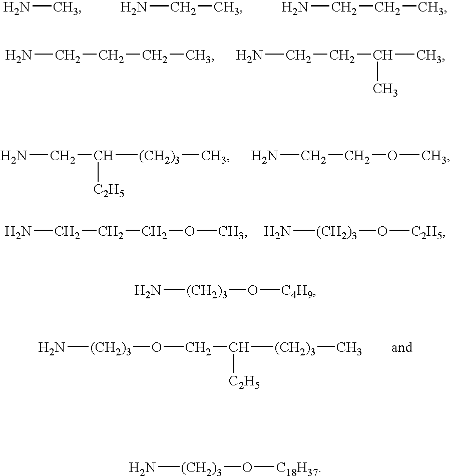

With particular advantage, however, the process of the invention is used for the preparation of N,N′-disubstituted 1,4-diarylaminoanthraquinones for which the aromatic amines are primary and correspond in particular to the formula

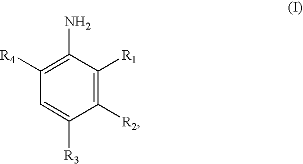

in which

R₁, R₃ and R4 independently of one another denote H or C₁-C₁₂-alkyl, especially C₁-C₄-alkyl, and

R₂ stands for H or —SO₂—NH-R₅, with R₅ standing for optionally substituted aryl, especially C₆-C₁₀-aryl, such as phenyl or naphthyl, or alkyl, especially C₁-C₄-alkyl, such as methyl, ethyl, propyl or butyl, and possible substituents being preferably C₁-C₄-alkyl, OH, halogen, C₁-C₄-alkoxy and C₆-C₁₀-aryloxy.

Particularly preferred aromatic amines of the formula (I) are those in which R₁ to R4 have the definitions given in the Table below.

TABLE

| R₁ | R₂ | R₃ | R₄ |
|---|---|---|---|
| H | H | CH₃ | H |
| H | H | t.-Bu | H |
| H | H | H | CH₃ |
| CH₃ | H | H | C₂H₅ |
| CH₃ | H | H | CH₃ |
| C₂H₅ | H | H | C₂H₅ |
| CH₃ | H | CH₃ | CH₃ |
| C₂H₅ | H | CH₃ | C₂H₅ |
| CH₃ | SO₂NH—C₆H₅ | CH₃ | CH₃ |

In one particularly preferred embodiment the process of the invention is conducted in the presence of boric acid. The latter is preferably employed in an amount of from 0.1 to 1 mole equivalent, based on the anthraquinone (total amount of quinizarine and leucoquinizarine) hydroxyl group to be replaced, in particular from 0.25 to 0.8 mole equivalent.

The process of the invention can of course also be carried out in the presence of additional condensation agents, such as are described, for example, in DE-A-2 342 469 (hydrochloric acid), U.S. Pat. No. 4,083,683 (acetic acid) or DE-A 195 17 071 (hydroxy carboxylic acids). The process of the invention is preferably conducted without such additions.

The process of the invention is preferably conducted at a temperature of from 60 to 200° C., more preferably from 90 to 160° C., in particular from 110 to 140° C.

Water is preferably removed during the reaction, for example by distillation with a water trap. The distillation may be carried out under atmospheric pressure, under reduced pressure or else with increased pressure.

The process of the invention proceeds with much greater selectivity than prior art processes. The gain in yield is particularly large when using aromatic amines of the formula (I). After the end of the condensation reaction of the invention, the reaction mixture generally possesses a temperature which lies preferably above the boiling point of water (more than 100° C.).

Temperatures below this are also advantageous where the external pressure has been reduced. After the end of the reaction the reaction mixture is preferably cooled.

For the oxidation of any leuco compounds present, it is preferred to pass air through the reaction mixture. Alternatively, oxidation can be carried out with oxidizing agents other than oxygen. Where appropriate, oxidation may also be omitted. Next, generally, the 1,4-diaminoanthraquinone compound, which is generally precipitated with aliphatic alcohols such as methanol, ethanol, propanol, butanol or with water or alcohol mixtures, is isolated. An advantage of the process of the invention is that the desired products are obtained in excellent yields and qualities even without precipitation with an aliphatic alcohol and/or water.

The 1,4-diaminoanthraquinone compound is preferably filtered and washed, preferably with the stated alcohols. This is generally followed by washing with water and, finally, by drying. Precipitation with aqueous hydrochloric acid as known from U.S. Pat. No. 4,083,683, Example 1, may also be used for isolation.

The yields specified in the Examples are based on the total amount of quinizarine and leucoquinizarine employed.

The process of the invention is distinguished by an improved process regime (e.g. low viscosity), by an excellent space-time yield, and by improved process products.

The dyes prepared by the process of the invention are especially suitable for mass coloration of plastics.

By mass coloration here are meant in particular processes in which the dye is incorporated into the melted polymer material, with the aid of an extruder, for example, or in which the dye is in fact added to starting components used to produce the polymer, e.g., to monomers prior to the polymerization.

Particularly preferred plastics are thermoplastics, example being vinyl polymers, polyesters, polyamides, and polyolefins, especially polyethylene and polypropylene, or polycarbonates.

Suitable vinyl polymers are polystyrene, styrene-acrylonitrile copolymers, styrene-butadiene copolymers, styrene-butadiene-acrylonitrile terpolymers, polymethyl methacrylate, polyvinyl chloride, etc.

Also suitable, inter alia, are polyesters such as polyethylene terephthalates, polycarbonates and cellulose esters, for example.

Preference is given to polystyrene, styrene copolymers, polycarbonates, polymethacrylates, and polyamides. Particular preference is given to polystyrene, polyethylene and polypropylene.

The polymer material mentioned may be present singly or in mixtures, as plastic compositions or as melts.

The dyes obtained by the process of the invention are employed preferably in finely divided form, it being possible but not mandatory to use dispersants. If the dye obtained by the process of the invention is used after polymerization, it is preferably ground or dry-mixed with the polymer granules and this mixture is plastified and homogenized, for example, on mixing rolls or in screws.

Alternatively, the dyes obtained can be added to the composition in liquid melt form and homogeneously distributed by stirring. The material precolored in this way is then processed further in the normal way, for example by spinning into bristles, filaments, etc. or by extrusion or by injection molding, to give shaped parts.

Since the dyes are resistant to polymerization catalysts, especially peroxides, it is also possible to add the dye to the monomeric starting materials for the plastics and then to carry out polymerization in the presence of polymerization catalysts. For this purpose the dye is preferably dissolved in or intimately mixed with the monomeric components.

The dyes obtained by the process of the invention are used preferably for dyeing the aforementioned polymers in amounts of from 0.0001 to 1% by weight, in particular from 0.01 to 0.5% by weight, based on the polymer amount.

By adding pigments which are insoluble in the polymers, such as titanium dioxide, for example, it is possible to obtain corresponding, valuable, hiding colorations.

Titanium dioxide can be used in an amount of from 0.01 to 10% by weight, preferably from 0.1 to 5% by weight, based on the polymer amount.

The dying process of the invention produces transparent or hiding, brilliant blue to green dyeings having good heat stability and also good light, weather and sublimation fastness.

In the dyeing process of the invention it is also possible to use mixtures of the dyes of the formula (I) with other dyes and/or organic and/or inorganic pigments. The invention is elucidated but not restricted by the following examples, in which the parts are given by weight; percentages are weight percentages (% by weight).

### EXAMPLES

### Example 1

165 ml of n-butanol are metered into a 500 ml 4-necked flask. With stirring, 50.5 g of p-toluidine, 15.5 g of boric acid and 42 g of 99.5% quinizarine are introduced. The mixture is heated at reflux with a water trap. After 10 minutes of reflux, 7.5 g of 93.5% dihydroquinizarine in solution in 22.5 ml of N-methyl-2-pyrrolidone are added dropwise over the course of 3 hours. After a further 9 hours at reflux the mixture is cooled and filtered and the filter product is washed with 80 ml of hot n-butanol at 60° C., then in portions with 200 ml of hot methanol at 60° C., thereafter with 2000 ml of hot water at 60° C., and is finally dried in vacuo.

Yield: 79 g (93.1% of theory)

In the mass coloration of polystyrene the product gives a very clear bluish green.

### Example 2

330 ml of n-butanol and 45 ml of N-methyl-2-pyrrolidone are metered into a 1000 ml sulphation vessel. With stirring, 101 g of p-toluidine, 31 g of boric acid and 84 g of 99.5% quinizarine and 15 g of 93.5% dihydroquinizarine are introduced. The mixture is heated at reflux with a water trap. After 5 hours at reflux the mixture is cooled and filtered and the filter product is washed with 160 ml of hot n-butanol at 60° C., then in portions with 400 ml of hot methanol at 60° C., thereafter with 4000 ml of hot water at 60° C., and finally is dried in vacuo.

Yield: 159 g (93.7% of theory)

In the mass coloration of polystyrene the product gives a very clear bluish green.

### Example 3

330 ml of n-butanol are metered into a 1000 ml sulphation vessel. With stirring, 45 g of caprolactam, 101 g of p-toluidine, 31 g of boric acid and 84 g of 99.5% quinizarine and 15 g of 93.5% dihydroquinizarine are introduced. The mixture is heated at reflux with a water trap. After 5 hours at reflux the mixture is cooled and filtered and the filter product is washed with 160 ml of hot n-butanol at 60° C., then in portions with 400 ml of hot methanol at 60° C., thereafter with 4000 ml of hot water at 60° C., and finally is dried in vacuo.

Yield: 159 g (93.7% of theory)

In the mass coloration of polystyrene the product gives a clear bluish green.

### Example 4

330 ml of n-butanol and 45 ml of N-methyl-2-pyrrolidone are metered into a 1000 ml sulphation vessel. With stirring, 101 g of p-toluidine, 31 g of boric acid, 1 g of p-toluenesulphonlc acid and 84 g of 99.5% quinizarine and 15 g of 93.5% dihydroquinizarine are introduced. The mixture is heated at reflux with a water trap. After 5 hours at reflux the mixture is cooled and filtered and the filter product is washed with 160 ml of hot n-butanol at 60° C., then in portions with 400 ml of hot methanol at 60° C., thereafter with 4000 ml of hot water at 60° C., and finally is dried in vacuo.

Yield: 154 g (90.7% of theory)

In the mass coloration of polystyrene the product gives a clear bluish green.

### Example 5

320 ml of n-butanol are metered into a 1000 ml sulphation vessel. With stirring, 80 g of caprolactam, 101 g of p-toluidine, 31 g of boric acid and 84 g of 99.5% quinizarine and 15 g of 93.5% dihydroquinizarine are introduced. The mixture is heated at reflux with a water trap. After 5 hours at reflux the mixture is cooled and filtered and the filter product is washed with 160 ml of hot n-butanol at 60° C., then in portions with 400 ml of hot methanol at 60° C., thereafter with 4000 ml of hot water at 60° C., and finally is dried in vacuo.

Yield: 153 g (90.2% of theory)

In the mass coloration of polystyrene the product gives a very clear bluish green.

### Example 6

240 ml of n-butanol are metered into a 1000 ml sulphation vessel. With stirring, 160 g of caprolactam, 101 g of p-toluidine, 31 g of boric acid and 84 g of 99.5% quinizarine and 15 g of 93.5% dihydroquinizarine are introduced. The mixture is heated at reflux with a water trap. After 6 hours at reflux the mixture is cooled and filtered and the filter product is washed with 160 ml of hot n-butanol at 60° C., then in portions with 400 ml of hot methanol at 60° C., thereafter with 4000 ml of hot water at 60° C., and finally is dried in vacuo.

Yield: 151 g (89.0% of theory)

In the mass coloration of polystyrene the product gives a very clear bluish green.

### Example 7

240 ml of toluene are metered into a 1000 ml sulphation vessel. With stirring, 360 g of caprolactam, 101 g of p-toluidine, 20 g of boric acid, 10 g of lactic acid and 84 g of 99.5% quinizarine and 15 g of 93.5% dihydroquinizarine are introduced. The mixture is heated at reflux with a water trap. After 5 hours at reflux the mixture is cooled and filtered and the filter product is washed in portions with 400 ml of hot methanol at 60° C., thereafter with 4000 ml of hot water at 60° C., and finally is dried in vacuo.

Yield: 127 g (74.8% of theory)

In the mass coloration of polystyrene the product gives a bluish green.

### Example 8

In a 1000 ml sulphation vessel, 400 g of caprolactam, 101 g of p-toluidine, 20 g of boric acid, 10 g of lactic acid and 84 g of 99.5% quinizarine and 15 g of 93.5% dihydroquinizarine are introduced. The mixture is heated at 110° C. After 5 hours the mixture is cooled and filtered, and the filter product is then washed in portions with 400 ml of hot methanol at 60° C and then with 4000 ml of hot water at 60° C., and finally is dried in vacuo.

Yield: 121 g (71.3% of theory)

In the mass coloration of polystyrene the product gives a bluish green.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. Process for the preparation of N,N′-disubstituted 1,4-diamino-anthraquinones, comprising reacting 1,4-dihydroxyanthraquinones with aliphatic or aromatic amines in the presence of a dipolar-aprotic solvent possessing a solubility in water of at least 30% by weight at 20° C., and at least one second solvent, different from the first.

2. Process according to claim 1, characterized in that the dipolar-aprotic solvent is a water-miscible solvent.

3. Process according to claim 1, characterized in that the dipolar-aprotic solvent is an optionally substituted formamide, sulphoxide, lactam or lactone.

4. Process according to claim 1, characterized in that the dipolar-aprotic solvent is dimethylformamide, dimethyl sulphoxide, butyrolactone, caprolactam or N-methyl-2-pyrrolidone.

5. Process according to claim 1, characterized in that the dipolar-aprotic compound is N-methyl-2-pyrrolidone.

6. Process according to claim 1, operated in the presence of boric acid.

7. Process according to claim 6, operated without the addition of mineral acids or carboxylic acids.

8. Process according to claim 1, characterized in that the aliphatic or aromatic amines are primary.

9. Process according to claim 1, characterized in that the aromatic amines correspond to the formula 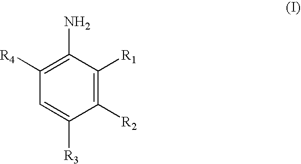
in which
R₁, R₃ and R4 independently of one another denote H or alkyl selected from the group consisting of C₁-C₁₂-alkyl, and
R₂ stands for hydrogen or —SO₂—NH-R₅, with R₅ standing for optionally substituted aryl or optinally substituted alkyl.

10. Process according to claim 9, characterized in that R₁, R₂ and R4 stand for H and R₃ stands for methyl.

11. Process according to claim 1, characterized in that the second solvent possesses a solubility in water of less than 20% by weight at 20° C.

12. Process according to claim 1, characterized in that the solubility of the second solvent in water lies between 5% by weight and 15% by weight at 20° C.

13. Process according to claim 1, characterized in that the second solvent forms an azeotrope with water.

14. Process according to claim 1, characterized in that the second solvent is miscible with the dipolar-aprotic solvent.

15. Process according to claim 1, characterized in that the second solvent is an aliphatic alcohol, especially n-butanol.

16. Process according to claim 1, characterized in that the dipolar-aprotic solvent and the second solvent are used in a ratio of from 5:95 to 50:50.

17. Process according to claim 1, in which 1,4-dihydroxyanthraquinone (quinizarine) is used in a mixture with its leuco form, 2,3-dihydro-1,4-dihydroxyanthraquinone (leucoquinizarine).

18. Process according to claim 17, in which the leuco form is not generated or added until during the course of the reaction.

19. A process for preparing plastics comprising coloring the plastics with the compound prepared by a process according to claim 1.
